(19) United States
(12) Patent Application Publication
(10) Pub. No.: US 2025/0320563 A1
(43) Pub. Date: Oct. 16, 2025

(54) SAMPLE PREPARATION FOR CELL-FREE DNA ANALYSIS

(71) Applicant: NUCLEIX LTD., Rehovot (IL)

(72) Inventor: Eran BRAM, Rehovot (IL)

(73) Assignee: NUCLEIX LTD., Rehovot (IL)

(21) Appl. No.: 18/868,090

(22) PCT Filed: May 22, 2023
(86) PCT No.: PCT/IB2023/000333

§ 371 Date: Nov. 21, 2024

Related U.S. Application Data
(60) Provisional application No. 63/344,625, filed on May 22, 2022.

(30) Foreign Application Priority Data
May 22, 2022 (IL) 293203

(51) Int. Cl. C12Q 1/6886; C12Q 1/6806; C12Q 1/683; C12Q 1/686; C12Q 1/6869

(52) U.S. Cl. CPC C12Q 1/6886; C12Q 1/6806; C12Q 1/683; C12Q 1/686; C12Q 1/6869; C12Q 2600/154

(57) ABSTRACT

Methods for the processing and analysis of blood samples obtained with blood collection tubes that reduce contamination of cfDNA by genomic DNA but that inhibit digestion by methylation-sensitive and/or methylation-dependent restriction enzymes.

This application claims the benefit of U.S. Provisional Application No. 63/344,625, filed May 22, 2022, and of Israeli Patent Application No. IL293203, filed May 22, 2022, from each of which priority is claimed and each of which is hereby incorporated by reference in its entirety including all tables, figures and claims.

### TECHNICAL FIELD

The invention is in the field of sample preparation for DNA methylation analysis.

### BACKGROUND

The following discussion of the background of the invention is merely provided to aid the reader in understanding the invention and is not admitted to describe or constitute prior art to the present invention.

Various techniques are known for analysing methylation of cytosine residues in DNA. One common method involves bisulfite conversion, in which unmethylated cytosines are converted to uracil using bisulfite. The converted DNA is then analysed, and a comparison of bisulfite-treated and bisulfite-untreated DNA reveals which cytosine residues were not converted to uracil (and thus were methylated). One major drawback with this technique is that bisulfite conversion is chemically harsh, leading to high levels of degradation of source material, which is a problem when using small quantities of source DNA. The chemical conversion is also biased, and inherently noisy.

Another technique uses a methylation-sensitive restriction enzyme (MSRE) whose activity is blocked if a cytosine in the enzyme's recognition sequence is methylated. Various MSRE-based techniques are available, using either single enzymes or combinations. For instance, the HELP assay uses a combination of HpaII and MspI. The recognition sequence for both of these enzymes is CCGG, but HpaII is methylation-sensitive. A comparison of the digestion products for the two enzymes can thus reveal which CCGG sites were methylated.

These enzyme-based techniques have also been used to analyse methylation of cell-free DNA (cfDNA), as in the EpiCheck platform marketed by Nucleix.

It is also possible to use a methylation-dependent restriction enzyme (MDRE) which digests its recognition sequence only if a cytosine is methylated i.e. the inverse of a MSRE-based assay.

The standardization of the pre-analytical phase is one of the major hurdles in incorporating cfDNA assays in clinical practice. While traditional EDTA tubes may be adequate for cfDNA applications in situations where it is possible to immediately process the blood samples to obtain the plasma component, in situations where samples must be stored for an extended period (e.g., when samples must be shipped to a central laboratory for processing) plasma cfDNA can become contaminated by genomic DNA originating from lysed or apoptotic cells present in the blood sample. Several companies have developed blood collection tubes that purport to stabilize blood cells and thereby limit this genomic DNA contamination. These include Streck Cell-Free DNA BCT® tubes, PAXgene Blood ccfDNA tubes, Roche Cell-Free DNA Collection tubes and Exact Sciences LBgard® blood tubes. The identity of the stabilization additives in these tubes is proprietary and so not generally published by the manufacturer, but the additives are typically divided into those that appear to contain or release aldehydes (e.g., acetaldehyde or formaldehyde) and those that are aldehyde-free. Although manufacturers may state that their tube does not impact cfDNA methylation analysis, the compatibility of these tubes may not be consistent across all analysis formats.

### SUMMARY

It is an object of the invention to provide methods for the preparation of cfDNA samples, and for the analysis of such samples, e.g., by methylation analysis. As described hereinafter, certain blood collection tubes can inhibit the digestion of cfDNA by certain restriction enzymes, and this inhibition is not resolved by increasing the amount of the enzyme(s) used in the digestion. As many cfDNA analysis formats rely on the use of such restriction enzymes, (e.g., use of methylation-sensitive restriction enzymes and/or one or more methylation-dependent restriction enzymes in assessing methylation status of the cfDNA), the methods described herein permit the use of these blood collection tubes that may be advantageous for collection, storage, and transport of blood samples without the need for immediate (e.g. within 4-8 hours) of collection or the use of low temperatures (e.g., 4° C.) to stabilize blood samples.

In a first aspect, the invention relates to methods for preparing a sample from a subject for methylation analysis. These methods comprise
processing a blood sample to obtain the plasma component of the blood sample, wherein the blood sample was collected using a blood collection tube comprising an anticoagulant and an agent that inhibits the release of genomic DNA from white blood cells in the sample into the plasma component of the blood sample;
isolating cell-free DNA (cfDNA) from the plasma component of the blood sample to provide a cfDNA sample; and
digesting the cfDNA sample with one or more methylation-sensitive restriction enzymes (MSREs) and/or one or more methylation-dependent restriction enzymes (MDREs) at a temperature between about 30° C. to about 45° C. for a digestion period of between about 8 hours to about 18 hours to provide a digested cfDNA sample, wherein less than 25% of the DNA molecules present in the cfDNA sample are single stranded DNA molecules during the digesting step.

As described hereinafter, the extended digestion period can provide a substantially complete digestion of the cfDNA sample by the MSREs and/or MDREs. The term “substantially complete digestion” as used herein refers to a point during the digestion period that the digestion plateaus, and no further digestion is occurring. This presumably indicates that the number of substrate digestion sites for the restriction enzymes being used no longer sufficient to support further reaction. In certain embodiments, the digestion period is between about 8 hours to about 11 hours, or between about 9 hours to about 10hours.

In certain embodiments it may be advantageous to inactivate the one or more MSREs and/or one or more MDREs following the digesting step to halt the digestion. By way of example, heating the digested cfDNA sample to about 65° C. for at least 20 minutes can result in such inactivation.

In certain embodiments it may be advantageous to reduce the amount of single-stranded DNA present in the cfDNA sample below the 25% level prior to digestion by the one or more MSREs and/or one or more MDREs. Thus, in certain embodiments less than 5% of the DNA molecules present in the cfDNA sample are single-stranded DNA molecules during the digesting step, or less than 5% of the DNA molecules present in the cfDNA sample are single-stranded DNA molecules during the digesting step. Extraction of cfDNA to obtain a cfDNA sample that minimizes the amount of single-stranded DNA present is described, for example, in WO2020/188561. In certain embodiments the cfDNA sample may be treated with a single-strand specific DNase to reduce the number of DNA molecules present in the cfDNA sample that are single-stranded DNA molecules. By way of example only, such a single-strand specific DNase is a Exonuclease I such as the E. coli ExoI sold commercially by New England Biolabs (catalog number M0293), and preferably a thermolabile Exonuclease I such as that sold commercially by New England Biolabs (catalog number M0568).

As demonstrated herein, certain blood collection tubes intended for cfDNA analysis can inhibit the digestion of cfDNA by MSREs and/or MDREs. Such blood collection tubes can comprise, for example, an agent that inhibits the release of genomic DNA from white blood cells such as formaldehyde, a formaldehyde-releasing reagent, or formalin. These blood collection tubes can also contain an anticoagulant such as potassium EDTA. In various embodiments, the use of the blood collection tube inhibits digestion of the cfDNA by the one or more MSREs and/or one or more MDREs as compared to the use of an ISO 6710: 1995 standard lavender closure EDTA blood collection tube, and this inhibition is not resolvable by increasing the concentration of the one or more MSREs and/or one or more MDREs.

In certain embodiments, the method further comprises amplifying at least one restriction locus in the digested cfDNA sample. The digesting step and the amplifying step may occur in separately different reaction vessels, or may preferably occur in the same reaction vessel. In either case, the one or more MSREs and/or one or more MDREs may be divalent cation-dependent, but the amount of divalent cation used in the digestion reaction may reduce the efficiency of the amplification reaction. In these embodiments, the free divalent cation concentration in the digested cfDNA sample is preferably reduced before the amplifying step. The concentration may be reduced by dilution, by adding a chelating agent, or by a combination of both.

In certain embodiments, the one or more MSREs and/or one or more MDREs are MSREs. Suitable MSREs for use in the invention include, but are not limited to, AatII, AccII, AciI, AcII, AfeI, AgeI, Aor13HI, Aor51HI, AscI, AsiSI, AvaI, BceAI, BmgBI, BsaAI, BsaHI, BsiEI, BsiWI, BsmBI, BspDI, BspT104I, BssHII, BstBI, BstUI, Cfr10I, ClaI, CpoI, DpnII, EagI, Eco52I, FauI, FseI, FspI, HaeII, HapII, HgaI, HhaI, HinP1I, HpaII, Hpy99I, HpyCH4IV, KasI, MluI, MspI, NaeI, NarI, NgoMIV, NotI, NruI, NsbI, PaeR7I, PluTI, PmaCI, PmlI, Psp1406I, PvuI, RsrII, SacII, SaII, ScrFI, SfoI, SgrAI, SmaI, SnaBI, SrfI, TspMI, ZraI. In preferred embodiments, the one or more MSREs and/or one or more MDREs comprise at least one MSRE selected from the group consisting of HinP1I and AciI, and in more preferred embodiments the one or more MSREs and/or one or more MDREs comprise, consist essentially of, or consist of, both HinP1I and AciI.

In related aspects, the present invention relates to methods for analysing cfDNA from a subject. These methods comprise:
preparing a digested cfDNA sample from the subject as described herein; and
performing one or more of the following analysis methods on the digested cfDNA: real time PCR on the digested cfDNA,
   sequencing of the digested cfDNA, including but not limited to NGS sequencing, and/or
   assessing methylation status of one or more CpG sites in cfDNA, such as by quantifying a degree of digestion at one or more of the one or more CpG sites.

In other related aspects, the present invention relates to methods for diagnosing the presence of absence of a cancer in a subject. These methods comprise:
preparing a digested cfDNA sample from the subject as described herein; and
assessing methylation status of one or more CpG sites in the digested cfDNA, wherein hypermethylation and/or hypomethylation of the one or more CpG sites is associated with the cancer.

In still other related aspects, the present invention relates to methods for treating or managing a cancer in a subject, comprising:
preparing a digested cfDNA sample from the subject as described herein; and
diagnosing a presence of cancer in the subject by assessing methylation status of one or more CpG sites in the digested cfDNA, wherein hypermethylation and/or hypomethylation of the one or more CpG sites is associated with the cancer; and
administering an anti-cancer treatment effective for the treatment of the cancer to the subject.

In further related aspects, the present invention relates to methods for collecting, transporting, and processing blood samples from a subject for cfDNA analysis, comprising:
collecting a blood sample from the subject at a first geographic location using a blood collection tube comprising an anticoagulant and an agent that inhibits the release of genomic DNA from white blood cells in the sample into the plasma component of the blood sample;
transporting the blood sample from the first geographic location collection to a second geographic location, wherein the sample is maintained at ambient temperature during transport;
preparing a digested cfDNA sample from the blood sample as described herein at the second geographic location.

The term “ambient temperature during transport” is not meant to indicate that, for example, the interior of a vehicle used for transport or an intermediate location through which the sample travels during transport are not air conditioned for the comfort of individuals involved in that transport. It is also not meant to indicate that an insulated shipping container is not used to prevent, for example, excessive heat or freezing of the sample. Rather, the term “ambient temperature during transport” as used herein refers to transporting the sample in the absence of any active heating or cooling being applied to the sample itself. So, for example, the blood sample is not shipped on ice or with another low temperature source such as a “cold pack” that maintains the sample between 2° C. and 8° C.

The methods described herein can permit the use of ambient temperature transport between a number of geographically dispersed locations that draw blood samples from individuals and a centralized processing laboratory by conventional overnight shipping methods. In certain embodiments, a time difference between collecting the blood sample at the first location and preparing the digested cfDNA sample at the second location is between about 8 hours and about 36 hours, between about 8 hours and about 24 hours, or at least about 12 hours.

### BRIEF DESCRIPTION OF THE FIGURES

FIG. 1 shows MSRE digestion levels, expressed as the sum of dCq across all tube/digestion protocols, for 11 samples under three sample processing conditions-blood collected in EDTA blood collection tubes, digestion for 2 hr; blood collected in cfDNA stabilization tubes, digestion for 2 hrs; blood collected in cfDNA stabilization tubes, digestion for 16 hrs.

FIG. 2 depicts the results from the 11 samples in FIG. 1 as a box-and-whisker plot.

FIG. 3 depicts the results from the 11 samples in FIG. 1 as a bar graph.

### DETAILED DESCRIPTION

The present invention, and the various features and advantageous details thereof, are explained more fully with reference to the non-limiting embodiments detailed in the following description. Descriptions of well-known components and techniques are omitted so as to not unnecessarily obscure the present invention. The examples used herein are intended merely to facilitate an understanding of ways in which the invention may be practiced and to further enable those of skill in the art to practice the invention. Accordingly, the examples should not be construed as limiting the scope of the invention.

### Methylation

The methods and compositions disclosed herein are useful for the analysis of DNA methylation, and in particular for analysing the presence or absence of 5-methyl modifications of cytosine in the context of a CG dinucleotide sequence (commonly denoted as ‘CpG’ dinucleotides or ‘CpG sites’) in eukaryotic DNA. CpG sites are not randomly distributed throughout eukaryotic genomes, and are frequently found in clusters known as ‘CpG islands’. These islands have been formally defined (Gardiner-Garden & Frommer (1987) J Mol Biol 196: 261-82) as regions which are at least 200bp long, having 50% or more GC content, and where the observed-to-expected CpG ratio is greater than 60% (i.e. where the number of CpG sites multiplied by the length of the sequence, divided by the number of C multiplied by the number of G, is greater than 0.6). CpG islands are often found near the start of a gene in mammalian genomes, and about 70% of promoters near transcription start sites in the human genome contain a CpG island. Methylation of multiple CpG sites within a promoter's CpG island is generally associated with stable silencing of gene expression from that promoter.

The human genome sequence contains around 28 million CpG sites (per haploid genome), with around 30,000 CpG islands. In any particular nucleated cell some CpG sites will be methylated and others will not. Patterns of methylation can differ between different cells and tissues within a subject, such that a specific CpG can be methylated in one cell or tissue but unmethylated in a different cell or tissue within the same subject.

It is known that tumors can display different methylation patterns compared to non-tumor cells (or compared to other types of tumor). Some sites can become hypermethylated in tumors, while others can become hypomethylated, and the difference in these patterns has been used to aid tumor diagnosis.

### Blood Collection

Blood can be collected in tubes that contain an anticoagulant and an agent to inhibit genomic DNA from blood cells in the sample being released into the plasma component of the blood sample. Such tubes are commercially available as glass cfDNA ‘Blood Collection Tubes’ or ‘BCT’ from Streck (La Vista, N E) e.g. as discussed by Diaz et al. (2016) PLOS One 11(11): e0166354, and they can stabilize cfDNA within blood for up to 14 days at 6-37° C. (thus providing advantages compared to typical K2EDTA collection tubes). Useful anticoagulants include, but are not limited to, EDTA, heparin, or citrate. Useful agents to inhibit release of genomic DNA from white blood cells include, but are not limited to, diazolidinyl urea, imidazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxy-methoxymethyl-1-laza-3,7-dioxabicyclo[3.3.0]octane, 5-hydroxymethyl-1-1aza-3,7dioxa-bicyclo[3.3.0]octane, 5-hydroxypoly[methyleneoxy]methyl-1-laza-3,7dioxabicyclo[3.3.0]-octane, quaternary adamantine, and mixtures thereof. Other useful components can include a quenching agent (e.g. lysine, ethylene diamine, arginine, urea, adenine, guanine, cytosine, thymine, spermidine, or any combination thereof) which can abate free aldehyde from reacting with DNA within a sample, aurintricarboxylic acid, metabolic inhibitors (e.g. glyceraldehyde and/or sodium fluoride), and/or nuclease inhibitors. For instance, a tube can include imidazolidinyl urea (or diazolidinyl urea), EDTA and glycine. Further information about suitable collection tubes can be found in WO2013/123030 and US2010/0184069.

Other useful collection tubes are available, including but not limited to various plastic tubes: the ‘Cell-Free DNA Collection Tube’ from Roche, made of PET; the ‘LBgard blood tube’ from Biomatrica, made from plastic and suitable for up to 8.5 mL of blood; and the ‘PAXgene Blood DNA tube’ from PreAnalytiX or Qiagen. These various tubes are discussed in more detail in Kerachian et al. (2021) Clinical Epigenetics 13,193 and Grölz et al. (2018) Current Pathobiology Reports 6: 275-86.

These various tubes can store up to 8.5 mL of blood, or sometimes up to 10 mL. A blood sample taken from a subject may thus typically have a volume of between 5-10 mL.

A 10 mL blood sample typically yields between 10-500 ng cfDNA, but can sometimes yield substantially higher amounts e.g. up to around 10 μg, particularly in certain cancer patients. Methods disclosed herein can be performed on the amount of cfDNA contained in a 10mL blood sample. Methods and compositions disclosed herein may typically use from 10-400 ng of cfDNA, for instance from 10-250 ng or from 10-200 ng.

Analysis of plasma-derived cfDNA is preferred. Kits for purifying cfDNA from plasma (and other bodily fluids) are readily available e.g. the MagMAX cfDNA isolation kit from ThermoFisher, the Maxwell RSC ccfDNA plasma kit from Promega, the Apostle MiniMax high efficiency isolation kit from Beckman Coulter, or the QIAamp or EZ1 products from Qiagen.

Methods and compositions disclosed herein may therefore utilise cfDNA extracted from a biological fluid sample of a subject, typically from a plasma or serum sample. Methods may begin with cfDNA which has already been prepared, or may include an upstream step of preparing the cfDNA. Similarly, methods may include an upstream step of obtaining a plasma sample before a step of preparing cfDNA from the plasma sample.

### Cell-Free DNA

The methods and compositions disclosed herein are particularly useful for analysing cell-free DNA (cfDNA) i.e. fragmented genomic DNA which is found in vivo in an animal within a bodily fluid rather than within an intact cell. The origin of cfDNA is not fully understood, but it is generally believed to be released from cells in processes such as apoptosis and necrosis. cfDNA is highly fragmented compared to intact genomic DNA (e.g. see Alcaide et al. (2020) Scientific Reports 10, article 12564), and in general circulates as fragments between 120-220 bp long, with a peak around 168bp (in humans).

cfDNA is present in many bodily fluids, including but not limited to blood and urine, and the methods and compositions disclosed herein can use any suitable source of cfDNA e.g. a blood sample (such as venous blood) or a urine sample. Ideally cfDNA is isolated from blood, and the blood may be treated to yield plasma (i.e. the liquid remaining after a whole blood sample is subjected to a separation process to remove the blood cells, typically involving centrifugation) or serum (i.e. blood plasma without clotting factors such as fibrinogen). Thus the methods and compositions disclosed herein can be used as part of so-called liquid biopsy testing, and can be implemented using plasma or serum cfDNA. Methods disclosed herein may thus include a step of purifying cfDNA from a blood, plasma or serum sample, to provide cfDNA for digestion and analysis. Methods may also include a step of obtaining a blood sample and preparing plasma or serum therefrom, thus providing a source for downstream purification of cfDNA.

Preferably, the cfDNA utilised in methods and composition disclosed herein is substantially free of single-stranded DNA (ssDNA) i.e. where less than 7% of the cfDNA molecules (by number) are single-stranded, and preferably less than 5% or less than 1% (i.e. such that at least 99% of the cfDNA molecules are double-stranded). In some embodiments, the cfDNA contains less than 0.1% ssDNA, less than 0.01% ssDNA, or may even contain no ssDNA (i.e. free of ssDNA). Extraction of cfDNA to obtain a cfDNA sample substantially free of ssDNA is described, for example, in WO2020/188561. Ensuring low levels of ssDNA avoids potential inhibition of restriction digestion, and also avoids undesired amplification of ssDNA. Commercial kits are available for quantifying single-stranded DNA in a sample e.g. the Promega QuantiFluor™ kit.

In some embodiments, all extracted cfDNA is used in the methods disclosed herein. In other embodiments, cfDNA is split into multiple fractions, and one or more fractions is not used in the methods disclosed herein but may instead be used in other analytical methods, or is kept for use in control experiments, or for other purposes.

In some embodiments, cfDNA is quantified prior to digestion (e.g. by weight, by concentration, etc.). In other embodiments, cfDNA is not quantified prior to digestion.

cfDNA used with the methods and compositions disclosed herein can be obtained from any eukaryotic subject, such as a mammal, and is ideally obtained from a human subject. In some embodiments the human subject may be known or suspected to have a disease (e.g. a cancer). In other embodiments the human subject may be known to be healthy. In some embodiments, the subject is not a pregnant woman.

### Restriction Enzymes and Digestion

Methods and compositions disclosed herein use restriction enzymes which recognise specific sequences in double-stranded DNA and introduce a double-stranded break into the DNA. The enzymes have a recognition site which contains a CpG sequence. Type II restriction enzymes are particularly useful i.e. enzymes where the double-stranded break is introduced within the recognition site. The use of multiple restriction enzymes permits simultaneous digestion in parallel within a sample.

More specifically, methods and compositions disclosed herein use methylation-sensitive restriction enzymes and/or methylation-dependent restriction enzymes. A MSRE cleaves the target DNA only if a CpG within its recognition site is unmethylated, and methylation inhibits the cleavage. Conversely, a MDRE cleaves the target DNA only if a CpG within its recognition site is methylated. MSREs and MDREs are readily available from well-known commercial suppliers, such as ThermoFisher, New England Biolabs, Promega, etc.

MSREs include, but are not limited to: AatII, AccII, AciI, AclI, AfeI, AgeI, Aor13HI, Aor51HI, AscI, AsiSI, AvaI, BceAI, BmgBI, BsaAI, BsaHI, BsiEI, BsiWI, BsmBI, BspDI, BspT104I, BssHII, BstBI, BstUI, Cfr10I, ClaI, CpoI, DpnII, EagI, Eco52I, FauI, FseI, FspI, HaeII, HapII, HgaI, HhaI, HinPII, HpaII, Hpy99I, HpyCH4IV, KasI, MluI, MspI, NaeI, NarI, NgoMIV, NotI, NruI, NsbI, PaeR7I, PluTI, PmaCI, PmII, Psp1406I, PvuI, RsrII, SacII, SaII, ScrFI, SfoI, SgrAI, SmaI, SnaBI, SrfI, TspMI, ZraI.

MDREs include, but are not limited to: BspEI, BtgZI, FspEI, GlaI, LpnPI, McrBC, MspJI, XhoI, XmaI.

Methods and compositions disclosed herein can comprise a plurality of restriction enzymes, wherein the plurality consists of MSRE and/or MDRE. Thus the plurality may include only MSREs, only MDREs, or a mixture of both (e.g. one or more MSRE plus one or more MDRE). In general, however, it is preferred to work with MSREs, without needing MDREs, and thus the plurality includes two or more MSREs. Using MSREs leads to cfDNA in which methylated CpG sites are intact but unmethylated CpG sites are digested. Thus, for any particular CpG-containing restriction site in a cfDNA sample, a higher percentage of methylation at this site leads to a lower extent of digestion compared to a cfDNA sample containing a higher percentage of methylation at this site.

A preferred plurality of MSREs includes both HinPII and AciI. In some embodiments it is possible to use one or more MSREs in addition to HinP1I and AciI, but it is more preferred to use HinP1I and AciI as the only two restriction enzymes for digestion of cfDNA. This pairing of enzymes covers over 99% of CpG islands in the human genome. With this MSRE pairing it is preferred to include HinP1I at an excess (measured in terms of enzymatic units) to AciI, and ideally an excess of at least 1.2:1 (i.e. at least 1.2 units of HinPII for every unit of AciI) e.g. at least 1.5:1, at least 1.75:1, at least 2:1, at least 3:1, at least 4:1, or at least 5:1. Ratios between 2:1 and 5:1 are particularly useful with human cfDNA, and an excess of about 4.5 is preferred. Digestion can be performed at about 37° C., until completion. Incubation at 37° C. for 2 hours is typically adequate for complete digestion of a cfDNA sample using HinP1I and AciI as described herein.

The concentration of restriction enzymes can be selected according to the particular experiments underway. Typically, HinPII can be used at 10-450 units per μg cfDNA, and AciI can be used at 2.5-100 units per μg cfDNA e.g. with a ratio of 4.5 units HinP1I per unit of AciI. In terms of solution concentration, HinP1I can be used at 35-45 units/ml, and AciI can be used at 5-15 units/mL cfDNA e.g. with a ratio of 4.5 units HinP1I per unit of AciI.

HinP1I (sometimes known as Hin6I) recognises the sequence GCGC and cleaves after the first G to leave a two nucleotide 5′ overhang (5′-G/CGC). It cuts well at 37° C. and can be heat-inactivated by heating at 65° C. for 20 minutes. For HinP1I, NEB recommends the use of its rCutSmart™ buffer (50 mM potassium acetate, 20 mM Tris-acetate, 10 mM magnesium acetate, 100 μg/mL recombinant albumin, pH 7.9). 1 unit of HinP1I is defined as the amount of enzyme required to digest 1 μg of λ DNA in 1 hour at 37° C. in a total reaction volume of 50 μl.

AciI recognises the sequence CCGC and cleaves after the first C to leave a two nucleotide 5′ overhang (5′-C/CGC). It cuts well at 37° C. and can be heat-inactivated by heating at 65° C. for 20 minutes. For AciI, NEB recommends the use of its rCutSmart™ buffer (50 mM potassium acetate, 20 mM Tris-acetate, 10 mM magnesium acetate, 100 μg/mL recombinant albumin, pH 7.9). 1 unit of AciI is defined as the amount of enzyme required to digest 1 μg of λ DNA in 1 hour at 37° C. in a total reaction volume of 50 μl. Its recognition site is non-palindromic.

λ DNA is a commonly used DNA substrate extracted from bacteriophage lambda (cI857ind 1 Sam 7), being 48502bp long. It is usually stored in 10 mM Tris-HCl (pH 8.0), 1 mM EDTA, and is widely available from commercial suppliers e.g. from NEB under catalogue number N3011S.

Because HinP1I and AciI share essentially the same conditions for digestion and inactivation they make a useful pairing for digesting DNA. In contrast, an enzyme such as HpaII requires heating to 80° C. for inactivation. BstUI and PvuI are not susceptible to heat inactivation. BstUI cuts optimally at 60° C. PvuI shows only 10% of its full activity in NEB's rCutSmart™ buffer.

After digestion it is preferred to inactivate the restriction enzymes, particularly if downstream amplification steps, such as PCR, will be used. Heat inactivation is particularly suitable, and HinPII and AciI can both be inactivated by heating the composition at 65° C. for at least 20 minutes e.g. for between 20-60 minutes. Further details about inactivation are given below.

Other useful combinations of enzymes comprise or consist of: (i) HinP1I+AciI+McrBC; (ii) HinPII+AciI+MspJI; (iii) HinPII+AciI+HpaII+HpyCH4IV+BstUI; (iv) HinP1I+AciI+HpaII+HpyCH4IV+AvaI; (v) MspJI+FspEI; (vi) MspJI+HinP1I+AciI; (vii) MspJI+FspEI+HinP1I+AciI; or (viii) MspJI+FspEI+HinP1I+AciI+HpyCH4IV.

MspJI shares essentially the same conditions for digestion and inactivation as HinP1I and AciI (e.g. it is active at 37° C. in rCutSmart™, and can be inactivated at 65° C.). This trio of enzymes can provide 85% CpG coverage and 100% CpG island coverage, so it is particularly useful.

Two further useful combinations comprise or consist of: (ix) HinP1I+AciI+HpaII; or (x) HinP1I+AciI+HpaII+HpyCH4IV. For these two combinations, methods and compositions of the invention should use at least one of the following additional features, as discussed elsewhere herein: (a) HinP1I is used at an excess to AciI in terms of enzymatic units; (b) digestion occurs for 11 hours or less; (c) the digested cfDNA is subjected to sequencing.

Where methods are described herein as involving “digestion”, this term (and also “digesting”, etc.) refers to the mixing of active restriction enzymes with DNA in conditions under which digestion can occur. If there are no recognition sites for the restriction enzyme in question (e.g. because it is a MSRE and all of the recognition sequences are fully methylated) then a step of “digestion” still takes place even though DNA cleavage does not occur.

### Methods

Various methods for digesting cfDNA using a combination of restriction enzymes (e.g. a combination of MSREs) are disclosed herein.

Enzymes and cfDNA are typically incubated for a long enough period for substantially complete digestion to occur i.e. further incubation does not lead to any measurable increase in cfDNA cleavage. For a typical sample collected in a cfDNA blood collection tube, the incubation time is lengthened relative to a sample collected in a standard lavender top EDTA blood collection tube. This can be achieved by incubation at between 30° C. and 45° C. (e.g., 37° C.) for 8 hours or more. Thus, in some embodiments, digestion may be performed for between 8-18 hours e.g. for between 8-10 hours or 9-10 hours.

After digestion has occurred, it is preferred to inactivate the restriction enzymes, particularly if downstream amplification steps will be used. HinP1I and AciI can both be inactivated by heating them to 65° C. e.g. by immersing the reaction mixture in a 65° C. water bath. Digestion reaction mixtures with cfDNA tend to have a low volume such that the temperature of the whole reaction mixture reaches 65° C. very quickly, leading to inactivation of the enzymes. In some embodiments heating at this temperature occurs for longer than 15 minutes, and ideally occurs for at least 20 minutes e.g. for 20-60 minutes. The temperature can exceed 65° C. if desired, but this is not required. This heating step is adequate for complete inactivation of the restriction enzymes i.e. such that the enzymes' digestion activity toward cleavable target cfDNA molecules under the digestion conditions employed prior to heating can no longer be measurably detected.

The invention also provides methods for analysing cfDNA, comprising digestion of cfDNA as discussed above, followed by downstream analytical steps e.g. a step of amplification (such as PCR, and in particular real-time PCR), a step of ligation (such as ligation of sequencing adapters), a step of DNA sequencing, etc. See further below.

The invention also provides methods for assessing methylation status of one or more CpG sites in cfDNA, comprising digestion of cfDNA as discussed above, followed by downstream analytical steps which quantify the degree of digestion at the one or more CpG sites. The degree of digestion may be determined individually for each site, or may be determined in aggregate.

The invention also provides methods for diagnosing the presence of absence of a cancer in a subject, comprising assessing methylation status of one or more CpG sites in cfDNA as discussed above, wherein hypermethylation and/or hypomethylation of the one or more CpG sites is associated with the cancer. In some embodiments, methods include a step of preparing a report in paper or electronic form based on the assessment of the presence or absence of the cancer, and optionally communicating the report to the subject and/or a healthcare provider of the subject.

The invention also provides a method for treating or managing a cancer in a subject, comprising diagnosing the presence of cancer as above, and administering a suitable anti-cancer treatment to the subject. The treatment may comprise one or more of surgical resection, chemotherapy, radiation therapy, immunotherapy, and/or targeted therapy.

Preferred methods do not include a step of bisulfite conversion. Other preferred methods include no step in which chemical changes are made to nucleobases within DNA e.g. no bisulfite conversion, no TAPS conversion, etc. TAPS conversion refers to TET-assisted pyridine borane sequencing.

Preferred methods do not use restriction enzyme isoschizomers, where one of the enzymes recognizes both the methylated and unmethylated forms of the restriction site while the other recognizes only one of these forms.

Preferred methods do not use a mixture of restriction enzymes in which at least one enzyme has a recognition sequence which includes a CpG but which is neither a MSRE or a MDRE i.e. an enzyme which digests regardless of the CpG methylation status.

### Compositions

Various compositions comprising a plurality of restriction enzymes (e.g. a plurality of MSREs) are disclosed herein. They are typically aqueous compositions comprising the enzymes in soluble active form, along with other components such as salts, buffers, co-factors, etc.

These compositions can include salts and/or buffers in aqueous solution. For instance, the composition can include 50 mM potassium acetate, 20 mM Tris-acetate, 10 mM magnesium acetate, 100 μg/mL recombinant albumin, pH 7.9 (i.e. the composition of the commercial rCutSmart™ buffer). As an alternative, the composition can include 50 mM Tris-HCl, 10 mM MgCl₂, 100 mM NaCl, 100 μg/mL recombinant albumin, pH 7.9 (i.e. the composition of the commercial NEBuffer™ r3.1 product). pH is measured at 25° C.

The compositions can include cfDNA, in particular when being used for digestion. As discussed above, in some compositions HinP1I is present at 10-450 units per μg cfDNA, and AciI is present at 2.5-100 units per μg cfDNA e.g. with a ratio of 4.5 units HinP1I per unit of AciI. In terms of solution concentration, HinP1I can be present at 35-45 units/ml, and AciI can be present at 5-15 units/mL cfDNA e.g. with a ratio of 4.5 units HinP1I per unit of AciI.

One useful composition of the invention thus comprises HinPII and AciI (e.g. with an excess of HinP1I, as described herein), potassium acetate, Tris-acetate, magnesium acetate, albumin, pH 7.8-8.0 (and, optionally, cfDNA to be digested). For instance, the composition may comprise from 4-5 units HinP1I, from 0.5-1.5 units AciI, 50 mM potassium acetate, 20 mM Tris-acetate, 10 mM magnesium acetate, 100 μg/mL albumin, pH 7.9, and cfDNA.

The restriction enzymes in the compositions are preferably present in enzymatically active form, as this permits their use to digest cfDNA. After digestion, however, the compositions can be heated (e.g. to 65° C.) to inactivate the enzymes, and so in some embodiments the restriction enzymes are present in heat-inactivated form.

In some embodiments, the compositions can also include PCR reagents e.g. suitable buffer/salt components (if required in addition to buffer/salt which persist after digestion), a DNA polymerase (such as a Taq polymerase), dNTPs, primers, probes, etc.

In some embodiments, the compositions can also include sequencing reagents e.g. one or more of sequencing adapters, DNA ligase (such as T4 ligase), Klenow fragment of DNA polymerase I, an A-tailing enzyme (such as Taq polymerase), a blunt-ending polymerase (such as T4 DNA polymerase), a kinase (such as T4 polynucleotide kinase), etc.

In some embodiments, the compositions can also include control DNA, as discussed below.

As noted above, when a composition includes HinP1I and AciI then HinP1I is ideally present at an excess (measured in terms of enzymatic units) to AciI, and ideally an excess of at least 1.2:1 e.g. at least 1.5:1, at least 1.75:1, at least 2:1, at least 3:1, at least 4:1, or at least 5:1. A ratio of at least 2:1 is often useful e.g. when the intention is to analyse with human cfDNA, and a ratio of about 4.5:1 has been found to be useful when digesting human cfDNA from plasma.

Preferred compositions do not include restriction enzyme isoschizomers, where one enzyme recognizes both the methylated and unmethylated forms of a restriction site and another recognizes only one of these forms.

Preferred compositions do not include a mixture of restriction enzymes in which at least one enzyme has a recognition sequence which includes a CpG but which is neither a MSRE or a MDRE i.e. an enzyme which digests regardless of the CpG methylation status.

### Downstream Amplification

After digestion, methods disclosed herein may include a step of amplification (e.g. PCR) performed on the digested cfDNA. Typically this amplification will be targeted to one or (preferably) more loci of interest e.g. loci containing CpG sites whose methylation status is known or expected to be associated with a particular biological state (e.g. with a cancer of interest). Thus upstream and downstream primers are used which flank the CpG site of interest, and the intervening CpG-containing sequence will be amplified if it has not been digested by restriction enzymes. The resulting amplicons can then be detected e.g. using a labelled probe which is complementary to a sub-sequence within the amplicons of interest.

Methods may therefore include a step of adding PCR reagents after digestion e.g. suitable buffer/salt components (if required in addition to buffer/salt remaining from digestion), a DNA polymerase (such as a Taq polymerase), dNTPs, primers and (optionally) probes. As an alternative, one or more of these components may be present during digestion e.g. it is possible to use a hot start PCR protocol, such that PCR reagents are already present during the digestion step but they do not become active until the reaction mixture is heated (e.g. during heat inactivation of the restriction enzymes).

Restriction digestion typically takes place in the presence of high levels of Mg⁺⁺. PCR usually relies on Mg⁺⁺, so standard PCR buffers include Mg⁺⁺. In this situation, however, addition of a standard PCR buffer can lead to an excess of Mg⁺⁺ which can inhibit efficiency of amplification. Thus added PCR reagents may include a lower level of Mg⁺⁺ than would normally be the case.

Where PCR primers and probes are present during MSRE digestion, they should be designed so that their sequences do not include the recognition site for the MSRE(s) which is/are being used.

Amplification and detection of amplicons may be carried out by conventional PCR using fluorescently-labeled primers followed by capillary electrophoresis of amplification products. In some embodiments, following amplification the amplification products are separated by capillary electrophoresis and fluorescent signals are quantified. An electropherogram plotting the change in fluorescent signals as a function of size (bp) or time from injection may be generated, wherein each peak in the electropherogram corresponds to the amplification product of a single locus. The peak's height (provided for example using “relative fluorescent units”, rFU) may represent the intensity of the signal from the amplified locus. Computer software may be used to detect peaks and calculate the fluorescence intensities (peak heights) of a set of loci whose amplification products were run on the capillary electrophoresis machine, and subsequently the ratios between the signal intensities.

A preferred PCR technique is real-time PCR (also known as qPCR), in which simultaneous amplification and detection of the amplification products are performed. Real-time PCR can be used with non-specific detection or sequence-specific detection. Non-specific detection (e.g. using a dsDNA-binding dye, such as SYBR Green) can be used within the methods disclosed herein, but is not ideal if it is desired to distinguish between multiple different amplicons in the same reaction. Thus it is more typical to use sequence-specific detection, and methods and compositions may use a labelled oligonucleotide probe (usually with a fluorophore and fluorescence quencher on the same probe, as in the TaqMan system) which is complementary to a specific sequence within nucleic acid amplicon(s) of interest. Different probes for amplicons derived from different target CpGs can be labelled with different fluorophores so that multiple different amplicons can be distinguished.

Real-time PCR may thus be achieved by using a hydrolysis probe based on combined reporter and quencher molecules. In such assays, oligonucleotide probes have a fluorescent moiety (fluorophore) attached to their 5′ end and a quencher attached to the 3′ end. During PCR amplification, the polynucleotide probes selectively hybridize to their target sequences on the template, and as the polymerase replicates the template it also cleaves the polynucleotide probes due to the polymerase's 5′-nuclease activity. When the polynucleotide probes are intact, the close proximity between the quencher and the fluorescent moiety normally results in a low level of background fluorescence. When the polynucleotide probes are cleaved, the quencher is decoupled from the fluorescent moiety, resulting in an increase of intensity of fluorescence. The fluorescent signal correlates with the amount of amplification products, i.e. the signal increases as the amplification products accumulate.

Suitable fluorophores include, but are not limited to, fluorescein, FAM, lissamine, phycoerythrin, rhodamine, Cy2, Cy3, Cy3.5, Cy5, Cy5.5, Cy7, FluorX, JOE, HEX, NED, VIC and ROX. Suitable fluorophore/quencher pairs are known in the art, including but not limited to: FAM-TAMRA, FAM-BHQ1, Yakima Yellow-BHQ1, ATTO550-BHQ2 and ROX-BHQ2.

Fluorescence may be monitored during each PCR cycle, providing an amplification plot showing the change of fluorescent signals from the probe(s) as a function of cycle number. In the context of real-time PCR, the following terminology is used:
“Quantification cycle” (“Cq”) refers to the cycle number in which fluorescence increases above a threshold, set automatically by software or manually by the user. In some embodiments, the threshold may be constant for each CpG locus of interest and may be set in advance, prior to carrying out the amplification and detection. In other embodiments, the threshold may be defined separately for each CpG locus after the run, based on the maximum fluorescence level detected for this locus during the amplification cycles.
“Threshold” refers to a value of fluorescence used for Cq determination. In some embodiments, the threshold value may be a value above baseline fluorescence, and/or above background noise, and within the exponential growth phase of the amplification plot.
“Baseline” refers to the initial cycles of PCR where there is little to no change in fluorescence.

Computer software is readily available for analysing amplification plots and determining baseline, threshold and Cq.

Where a CpG site has not been digested, and is thus amplified in subsequent PCR, relatively low Cq values are seen because detectable amplification products accumulate after a relatively small number of amplification cycles. Conversely, if amplicons are present at lower levels (e.g. because some CpG loci of interest were digested) then fewer amplicons are seen, and the Cq value is higher.

These results can thus indicate, for any given CpG site, the proportion of cfDNA molecules in a sample which were methylated/unmethylated at that CpG site. These figures can be expressed as a percentage, a fraction, a normalised value, etc.

Primers may vary in length, depending on the particular assay format and the particular needs. In some embodiments, the primers may be at least 15 nucleotides long, such as between 15-25 nucleotides or 18-25 nucleotides long. The primers may be adapted to be suited to a chosen amplification system.

Primers may be designed to generate amplicons between 60-150 bp long (when the relevant CpG site(s) is/are intact) e.g. between 70-140 bp long.

Oligonucleotide probes may vary in length. In some embodiments, the probes may include between 15-30 nucleotides, from 20-30 nucleotides, or from 25-30 nucleotides.

The oligonucleotide probes may be designed to bind to either strand of the double-stranded amplicons. Additional considerations include the melting temperature of the probes, which should preferably be comparable to that of the primers.

Where multiple CpG sites are analysed in parallel, with simultaneous amplification of more than one target in the same reaction mixture (co-amplification) using different primer pairs for each CpG site of interest, these different primers may be designed such that they can work at the same annealing temperature during amplification. Thus primers with similar melting temperature (Tm) can be designed e.g. within ±3°-5° C. of each other. Similar considerations apply where multiple probes are used.

Computer software is readily available for routine designing of primers and probes which meet the various requirements of any particular experiment.

### Downstream Sequencing

After digestion, methods disclosed herein may include a step of DNA sequencing, such as a step using next-generation sequencing ('NGS') techniques (also known as high-throughput sequencing). NGS generally involves three basic steps: library preparation; sequencing; and data processing. Examples of NGS techniques include sequencing-by-synthesis and sequencing-by-ligation (employed, for example, by Illumina Inc., Life Technologies Inc., PacBio, and Roche), nanopore sequencing methods and electronic detection-based methods such as Ion Torrent™ technology (Life Technologies Inc.). NGS may be performed using various high-throughput sequencing instruments and platforms, including but not limited to: Novaseq™, Nextseq™ and MiSeq™ (Illumina), 454 Sequencing (Roche), Ion Chef™ (ThermoFisher), SOLID® (ThermoFisher) and Sequel II™ (Pacific Biosciences). Appropriate platform-designed sequencing adapters are used for preparing the sequencing library, and are readily available from the platforms' manufacturers.

Library preparation for the major high-throughput sequencing platforms involves ligation of specific adapter oligonucleotides, also termed “sequencing adapters”, to the DNA fragments to be sequenced. Sequencing adapters typically include platform-specific sequences for fragment recognition by a particular sequencer e.g. sequences that enable ligated molecules to bind to the flow cells of Illumina platforms (e.g. the P5 and P7 sequences). Each sequencing instrument provider typically sells a specific set of sequences for this purpose. Further details of library preparation are discussed below.

Sequencing adapters can include sites for binding to a universal set of PCR primers. This permits multiple adapter-ligated DNA molecules to be amplified in parallel by PCR, using a single set of primers.

Sequencing adapters can include sample indices, which are sequences that enable multiple samples to be combined, and then sequenced together (i.e. multiplexed) on the same instrument flow cell or chip. Each sample index, typically 6-10 nucleotides, is specific to a given sample and is used for de-multiplexing during downstream data analysis to assign individual sequence reads to the correct sample. Sequencing adapters may contain single or dual sample indexes depending on the number of libraries combined and the level of accuracy desired.

Sequencing adapters can include unique molecular identifiers (UMIs) to provide molecular tracking, error correction and increased accuracy during sequencing. UMIs are short sequences, typically 5 to 20 bases in length, used to uniquely identify original molecules in a sample library. As each nucleic acid in the starting material is tagged to provide a unique molecular barcode, bioinformatics software can filter out duplicate reads and PCR errors with a high level of accuracy and report unique reads, removing the identified errors before final data analysis.

In some embodiments, sequencing adapters include both a sample barcode sequence and a UMI.

In some embodiments, sequencing adapters allow for paired-end sequencing.

In some embodiments, the compositions and methods disclosed herein use Y-shaped sequencing adapters i.e. adapters consisting of two single-stranded oligonucleotides which anneal to provide a double-stranded stem and two single-stranded ‘arms’. In other embodiments, the compositions and methods disclosed herein use hairpin sequencing adapters i.e. a single-stranded oligonucleotide whose 5′ and 3′ termini anneal to provide a double-stranded stem. For both Y-shaped and hairpin adapters the double-stranded stem can include a short single-stranded overhang e.g. a single A or T nucleotide. For both Y-shaped and hairpin adapters the double-stranded stem can be ligated to a cfDNA fragment, to prepare a sequencing library.

Suitable sequencing adapters for use in the compositions and methods disclosed herein may thus be TruSeq™ or AmpliSeq™ or TruSight™ adapters (for use on the Illumina platform) or SMRTbell™ adapters (for use on the PacBio platform).

Where sequencing adapters are added by ligation, this usually occurs at both ends of the DNA to be sequenced.

Restriction digestion can leave blunt-ends, but typically produces a single-stranded overhang. Library preparation steps can either preserve this overhang (i.e. add complementary nucleotides) or remove it. As the sequence of a post-digestion terminal single-stranded overhang can include useful information then it is preferred to add sequencing adapters in a way which preserves the overhang e.g. using enzymatic ligation in which a ligase enzyme covalently links a sequencing adapter to a DNA fragment where the terminal sequence of the adapter is complementary to the terminal sequence obtained using the restriction enzyme, or by using a polymerase to add complementary nucleotides and generate a blunt-ended fragment.

In addition to removing or filling in single-strand overhangs, end repair methods can be carried out before adapter ligation can ensure that DNA molecules contain 5′ phosphate and 3′ hydroxyl groups.

For some libraries, incorporation of a non-templated deoxyadenosine 5′-monophosphate (dAMP) onto the 3′ end of blunted DNA fragments is used in library preparation (a process known as dA-tailing). dA-tails prevent concatemer formation during downstream ligation steps and enable DNA fragments to be ligated to adapter oligonucleotides with complementary dT-overhangs.

As noted above, restriction digestion typically takes place in the presence of high levels of Mg⁺⁺. Sequencing library preparation may also rely on Mg⁺⁺, so standard library prep buffers include Mg⁺⁺. In this situation, however, addition of a standard library prep buffer can lead to an excess of Mg⁺⁺ which can inhibit efficiency of downstream steps. Thus added reagents may include a lower level of Mg⁺⁺ than would normally be the case for library preparation.

As an alternative approach to using lower levels of Mg⁺⁺, it is possible to add a chelating agent after digestion, which can remove the need for removal or dilution of excess Mg⁺⁺ for downstream amplification step(s). It has been found that the addition of a chelating agent at the concentrations disclosed herein impairs neither such amplification step(s) nor subsequent sequencing. The chelating agent can be added to provide an amplification reaction mix comprising the chelating agent and a divalent cation at a molar ratio of between 1:20 to 2:1. For instance, the reaction mix may include 8-20 mM Mg⁺⁺ e.g. about 10 mM magnesium. For instance, amplification may be carried out in a reaction mix comprising between 3-4 mM chelating agent and 4 mM Mg⁺⁺. The chelating agent may comprise one or both of EDTA and EGTA.

After library preparation, the prepared DNA molecules can be sequenced, to provide a plurality of “sequence reads”. These sequence reads are then subjected to data processing e.g. to remove sequences which do not fulfil desired quality criteria, to remove duplicates, to correct sequencing errors, to map sequences onto a reference genome, to count the number of sequence reads, etc. Computer software is readily available for performing these steps.

Any particular CpG site can feature in multiple sequence reads, which can be sequence reads derived from the same original cfDNA molecule and/or from different cfDNA molecules which span the same CpG site. Sequencing is suitably performed such that CpG site(s) of interest is/are seen in at least 100 sequence reads e.g. in at least 200, 300, 400, 500, 600, 700 or more sequence reads.

Sequence reads can be mapped to a reference genome i.e. a previously identified genome sequence, whether partial or complete, assembled as a representative example of a species or subject. A reference genome is typically haploid, and typically does not represent the genome of a single individual of the species but rather is a mosaic of the genomes of several individuals. A reference genome for the methods of the present invention is typically a human reference genome e.g. a complete human genome, such as the human genome assemblies available at the website of the National Center for Biotechnology Information or at the University of California, Santa Cruz, Genome Browser. An example of a suitable reference genome for human studies is the ‘hg18’ genome assembly. As an alternative, the more recent GRCh38 major assembly can be used (up to patch p13).

Mapping aligns sequence reads to the reference genome, to identify the location of the reads within the reference genome. The sequence reads that align are designated as being “mapped”. The alignment process aims to maximize the possibility for obtaining regions of sequence identity across the various sequences in the alignment, allowing mismatches, indels and/or clipping of some short fragments on the two ends of the reads. The number of sequence reads mapped to a certain genomic locus is referred to as the “read count” or “copy number” of this genomic locus. It is not necessary to map all sequence reads which are obtained; indeed, it is not unusual that a portion of sequence reads obtained in any given experiment will not be mappable.

The term “genomic locus” refers to a specific location within the genome, and may include a single position (a single nucleotide at a defined position in the genome) or a stretch of nucleotides starting and ending at defined positions in the genome. The specific position(s) may be identified by the molecular location, namely, by the chromosome and the numbers of the starting and ending base pairs on the chromosome. A genomic locus of interest herein contains at least one CpG site.

Where restriction digestion used a MSRE, sequence reads which span a particular CpG site are derived from molecules which were not digested i.e. which (with complete digestion) were methylated at that CpG site. The methylation level of this CpG site can be calculated by dividing its read count by an expected read count of this site (e.g. the read count which would be expected if it was fully methylated, and thus undigested). The expected read count may be determined using, for instance: (i) the read count of a control locus that is not cut by the restriction endonuclease; (ii) the average read count of a plurality such control loci; or (iii) the read count of the same CpG site in an undigested control sample, optionally corrected for sequencing depth differences.

As an alternative, the expected read count for a CpG site may be determined as the sum of the read count at this CpG site (indicating methylation) plus the sum of the read counts whose termini map to this CpG site (indicating non-methylation), taking account where necessary of any end-repair which took place during library preparation.

To avoid double-counting, the non-methylated CpG sites can be taken as sequencing reads whose 5′ ends map to a site, as sequencing reads whose 3′ ends map to a site, or as the half of the sum of sequencing reads whose 5′ ends or 3′ ends map to a site. As some library preparation methods can result in depletion of small fragments, which are then not sequenced (e.g. in CpG islands, where a starting cfDNA molecule is cleaved by a MSRE at more than one unmethylated site, thus providing 3 or more restriction fragments, some of which are very small), the observed number of unmethylated CpG sites may be lower than the true value in the original sample. This distortion can be somewhat addressed by using the larger of the number of reads whose 3′ ends map to a site and the number of reads whose 5′ ends map to a site (or to use the mean).

These calculations can thus provide, for any given CpG site, the proportion of cfDNA molecules in a sample which were methylated at that CpG site. Conversely, similar calculations can provide the proportion of a particular CpG site which were unmethylated. These figures can be expressed as a percentage, a fraction, a normalised value, etc.

One way of expressing coverage of a particular CpG site is referred to as ‘Hitspan 100’, which refers to the number of sequence reads which span a certain CpG position with at least 50 nucleotides both upstream and downstream. For example, a Hitspan100 of 90 at a specific CpG site means that there are 90 sequence reads which span this site with at least 50 nucleotides both upstream and downstream.

Methods disclosed herein do not require differential adapter tagging of methylated vs. unmethylated DNA molecules. The same population of adapters can be used for all molecules.

### Controls

Methods disclosed herein can take advantage of positive and negative controls. In some embodiments, parallel analysis can be performed on one or more of:
A DNA control which does not contain a recognition sequence for the restriction enzymes used for digestion. If this DNA is digested, this indicates that the method has not performed correctly.
A DNA control which contains a fully methylated recognition sequence for the restriction enzymes used for digestion. If this DNA is digested when a method uses only MSREs, this indicates that the method has not performed correctly (and conversely for MDREs).
A DNA control which contains a fully unmethylated recognition sequence for the restriction enzymes used for digestion. If this DNA is not fully digested when a method uses only MSREs, this indicates that the method has not performed correctly (and conversely for MDREs).

These DNA controls can also be used as a reference point for analysis, for checking completeness of digestion, etc. As mentioned above, for instance, if fragments are obtained using MSRE digestion then it can be useful in a downstream NGS experiment to know the expected read count, and one way of obtaining this value is to look at the read count for DNA which does not contain the recognition sequence for the MSRE, or at the read count for DNA which contains the recognition sequence but is fully methylated.

For these purposes, it is preferred that the DNA control should be similar in size and composition to cfDNA molecules which contain CpG sites of interest. Thus, although it is possible to use synthetic DNA or PCR amplicons or bacterial plasmid DNA as an unmethylated control, these are more useful if they have sizes which are similar to cfDNA (e.g. a long synthetic DNA, or an appropriately-sized restriction fragment prepared from a plasmid).

Control experiments can be performed internally in a sample, or externally. For an internal control, control DNA can be present in a sample already (e.g. cfDNA containing a CpG site which is known to be ubiquitously (un) methylated, or cfDNA which does not contain a recognition sequence for the restriction enzymes being used) and/or can be added (e.g. synthetic DNA, added to cfDNA). The control DNA can therefore be processed in combination with the cfDNA, and experiences the same conditions as the cfDNA, and so a method can involve co-amplification of a restriction locus and a control locus. For an external control, control DNA is subjected to the same treatment as the cfDNA but not as part of the same reaction mixture.

Thus control DNA, like cfDNA, can be digested with restriction enzymes and then subjected to downstream analytical steps e.g. amplification, DNA sequencing, etc. Real-time PCR of suitable control loci can give a result that can be used as a reference point. For instance, the signals obtained from cfDNA at a CpG site of interest and from control DNA (in particular, from control DNA which is not digested by the restriction enzymes being used) can be compared, and the signal ratio can be used to determine the degree of methylation at a CpG site of interest, because the ratio of signal reflects the ratio of methylation. Thus methods disclosed herein can be performed without requiring evaluation of absolute methylation levels at genomic loci, but rather by calculating a signal ratio between the analyzed genomic loci and a control. This contrasts with some conventional methods of methylation analysis for distinguishing between tumor-derived and normal DNA, which require determining actual methylation levels at specific genomic loci. The methods disclosed herein can thus eliminate the need for standard curves and/or additional laborious steps involved in determination of absolute methylation levels, thereby offering a simple and cost-effective procedure. An additional advantage when using an internal control is that signal ratios are obtained for loci amplified in the same reaction mixture under the same reaction conditions, which can help to eliminate sources of potential error (e.g. the potential for differences between reaction mixtures, such as the concentration of template, enzyme, etc.).

Methods which use qPCR may therefore involve calculating signal intensity ratios between a CpG site co-amplified after digestion of DNA as disclosed herein, thereby providing a methylation status for the CpG site. This methylation status can then be compared to reference values (e.g. obtained from healthy subjects, or from subjects having a known disease) and, based on the comparison, a diagnostic result can be derived. Thus a method may involve: co-amplifying from restriction endonuclease-digested DNA a CpG site and a control locus, thereby generating co-amplification products; determining a signal intensity for each generated co-amplification product; and calculating a ratio between the signal intensities of the co-amplification products of the CpG site and the control locus.

The ratio between the signal intensities of the co-amplification products may be calculated by determining the quantification cycle (Cq) for each locus and calculating 2^{(Cq control locus-Cq CpG site)}. In other words, the reduction in Cq relative to the control locus is determined, and this value is used as the exponent of 2 to calculate the ratio.

Thus, using qPCR or sequencing, it is possible, based on the degree of digestion at any particular CpG site, to derive a numerical value which represents the degree of methylation of that CpG site in a cfDNA sample. This value may be expressed in a variety of ways e.g. as a ratio or percentage of the cfDNA molecules that are methylated at a CpG site, or as an intensity of a signal obtained from a particular CpG site, or as the ratio between a CpG site and a control locus, etc.

### Systems and Kits

The invention also provides various systems and kits.

A system can comprise computer processor(s) for performing and/or controlling the methods disclosed herein, and/or for processing the results e.g., for performing calculations based on the results. Methods which are at least partially computer-implemented are provided.

A system or kit may comprise: a blood, plasma or serum sample of a human subject; components for carrying out a method disclosed herein on at least one CpG site; and computer software stored on a non-transitory computer readable medium, the computer software being able to direct a computer processor to determine a methylation value for the at least one CpG locus based on the methylation assay. The software may also be able to link the methylation value to a diagnostic result or prediction e.g. by comparing one or more methylation value(s) to one or more reference values to assess the presence of a disease in the subject. The computer software may receive data from a qPCR and/or a NGS experiment.

Components for carrying out a method disclosed herein encompass biochemical components (e.g., enzymes, primers, probes, NTPs, etc.), chemical components (e.g., buffers, reagents), and technical components (e.g., a PCR system, such as a real-time PCR system, and equipment such as tubes, vials, plates, pipettes).

The system may be able to prepare and/or communicate a report to the subject and/or to a healthcare provider of the subject, based on the methylation values.

Computer software includes processor-executable instructions that are stored on a non-transitory computer readable medium. The computer software may also include stored data. The computer readable medium is a tangible computer readable medium, such as a compact disc (CD), magnetic storage, optical storage, random access memory (RAM), read only memory (ROM), or any other tangible medium.

Computer-related methods and steps described herein are implemented using software stored on non-volatile or non-transitory computer readable instructions that when executed configure or direct a computer processor or computer to perform the instructions.

Each of the system, server, computing device, and computer described in this application can be implemented on one or more computer systems and be configured to communicate over a network. They all may also be implemented on one single computer system. In one embodiment, the computer system includes a bus or other communication mechanism for communicating information, and a hardware processor coupled with bus for processing information.

A computer system also includes a main memory, such as a random-access memory (RAM) or other dynamic storage device, coupled to bus for storing information and instructions to be executed by processor. Main memory also may be used for storing temporary variables or other intermediate information during execution of instructions to be executed by processor. Such instructions, when stored in non-transitory storage media accessible to processor, render computer system into a special-purpose machine that is customized to perform the operations specified in the instructions.

A computer system can include read only memory (ROM) or other static storage device coupled to bus for storing static information and instructions for processor. A storage device, such as a magnetic disk or optical disk, is provided and coupled to bus for storing information and instructions.

A computer system may be coupled via bus to a display, for displaying information to a computer user.

An input device, including alphanumeric and other keys, can be coupled to bus for communicating information and command selections to processor. Another type of user input device is cursor control, such as a mouse, a trackball, or cursor direction keys for communicating direction information and command selections to processor and for controlling cursor movement on display.

Methods disclosed herein may be performed by a computer system in response to the processor executing one or more sequences of one or more instructions contained in main memory. Such instructions may be read into main memory from another storage medium, such as storage device. Execution of the sequences of instructions contained in main memory causes the processor to perform the process steps described herein. In alternative embodiments, hard-wired circuitry may be used in place of or in combination with software instructions.

Suitable storage media include any non-transitory media that store data and/or instructions that cause a machine to operation in a specific fashion. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media are distinct from, but may be used in conjunction with, transmission media. Transmission media participates in transferring information between storage media. For example, transmission media includes coaxial cables, copper wire and fiber optics, including the wires that comprise bus.

The invention also provides a kit comprising: (i) a composition comprising a plurality of restriction enzymes, as discussed above; and (ii) components for analysing cfDNA which has been digested with the composition. These components may be e.g. components for performing PCR, or for preparing a sequencing library from digested cfDNA. For instance, the kit may include one or more of: (a) a buffer solution e.g. with 50 mM potassium acetate, 20 mM Tris-acetate, 10 mM magnesium acetate, 100 μg/mL recombinant albumin, pH 7.9, or with 50 mM Tris-HCl, 10 mM MgCl₂, 100 mM NaCl, 100 μg/mL recombinant albumin, pH 7.9; (b) a DNA polymerase, dNTPs, primers and, optionally, one or more probes; (c) sequencing adapters; (d) an enzyme solution, including a DNA ligase and/or a DNA polymerase; and/or (e) control DNA Further details of these components (a) to (e) are discussed elsewhere herein.

A kit may include an instruction manual for carrying out the methods as disclosed herein.

A kit may include a non-transitory computer readable medium storing a computer software comprising instructions that when executed configure or direct a computer processor to perform the method steps disclosed herein.

### Preferred Embodiments

The following are preferred embodiments of the invention:

Embodiment 1. A method of preparing a sample from a subject for methylation analysis, comprising:
processing a blood sample to obtain the plasma component of the blood sample, wherein the blood sample was collected using a blood collection tube comprising an anticoagulant and an agent that inhibits the release of genomic DNA from white blood cells in the sample into the plasma component of the blood sample;
isolating cell-free DNA (cfDNA) from the plasma component of the blood sample to provide a cfDNA sample; and
digesting the cfDNA sample with one or more methylation-sensitive restriction enzymes (MSREs) and/or one or more methylation-dependent restriction enzymes (MDREs) at a temperature between about 30° C. to about 45° C. for a digestion period of between about 8hours to about 18 hours to provide a digested cfDNA sample, wherein less than 25% of the DNA molecules present in the cfDNA sample are single stranded DNA molecules during the digesting step.

Embodiment 2. A method according to embodiment 1, wherein the digestion period is between about 8 hours to about 11 hours.

Embodiment 3. A method according to embodiment 1, wherein the digestion period is between about 9 hours to about 10 hours.

Embodiment 4. A method according to one of embodiments 1-3, further comprising inactivating the one or more MSREs and/or one or more MDREs following the digesting step to halt the digestion.

Embodiment 5. A method according to embodiment 4, wherein the inactivating comprises heating the digested cfDNA sample to about 65° C. for at least 20 minutes.

Embodiment 6. A method according to one of embodiments 1-5, wherein less than 5% of the DNA molecules present in the cfDNA sample are single stranded DNA molecules during the digesting step.

Embodiment 7. A method according to one of embodiments 1-5, wherein less than 1% of the DNA molecules present in the cfDNA sample are single stranded DNA molecules during the digesting step.

Embodiment 8. A method according to one of embodiments 1-7, wherein the cfDNA sample is treated with a single-strand specific DNase to reduce the number of DNA molecules present in the cfDNA sample that are single stranded DNA molecules.

Embodiment 9. A method according to embodiment 8, wherein the single-strand specific DNase is an Exonuclease I.

Embodiment 10. The method according to one of embodiments 1-9, wherein the use of the blood collection tube inhibits digestion of the cfDNA by the one or more MSREs and/or one or more MDREs as compared to the use of an ISO 6710: 1995 standard lavender closure EDTA blood collection tube.

Embodiment 11. The method of embodiment 10, wherein the inhibition of digestion of the cfDNA is not resolvable by increasing the concentration of the one or more MSREs and/or one or more MDREs.

Embodiment 12. The method according to one of embodiments 1-11, wherein the method further comprises amplifying at least one restriction locus in the digested cfDNA sample.

Embodiment 13. The method according to embodiment 12, wherein the digesting step and the amplifying step occur in the same vessel.

Embodiment 14. The method according to embodiment 13, wherein the one or more MSREs and/or one or more MDREs are divalent cation-dependent, and the free divalent cation concentration in the digested cfDNA sample is reduced before the amplifying step.

Embodiment 15. The method according to embodiment 14, wherein the free divalent cation concentration is reduced by dilution.

Embodiment 16. The method according to embodiment 14, wherein the free divalent cation concentration is reduced by adding a chelating agent.

Embodiment 17. The method according to one of embodiments 1-16, wherein the one or more MSREs and/or one or more MDREs comprise one or more of AciI, HinP1I, and HhaI.

Embodiment 18. The method according to one of embodiments 1-17, wherein the agent that inhibits the release of genomic DNA from white blood cells comprises formaldehyde, a formaldehyde-releasing reagent, or formalin.

Embodiment 19. The method according to one of embodiments 1-18, wherein the anticoagulant is potassium EDTA.

Embodiment 20. A method for analysing cfDNA from a subject, comprising:
preparing a digested cfDNA sample from the subject according to one of embodiments 1-19; and
performing real time PCR on the digested cfDNA.

Embodiment 21. A method for analysing cfDNA from a subject, comprising:
preparing a digested cfDNA sample from the subject according to one of embodiments 1-19; and
sequencing of the digested cfDNA.

Embodiment 22. A method for assessing methylation status of one or more CpG sites in cfDNA, comprising:
preparing a digested cfDNA sample from the subject according to one of embodiments 1-19;
quantifying a degree of digestion at one or more of the one or more CpG sites.

Embodiment 23. A method for diagnosing the presence of absence of a cancer in a subject, comprising:
preparing a digested cfDNA sample from the subject according to one of embodiments 1-19;
assessing methylation status of one or more CpG sites in the digested cfDNA, wherein hypermethylation and/or hypomethylation of the one or more CpG sites is associated with the cancer.

Embodiment 24. A method for treating or managing a cancer in a subject, comprising:
diagnosing a presence of cancer in the subject by the method of embodiment 23; and
administering an anti-cancer treatment effective for the treatment of the cancer to the subject.

Embodiment 25. A method for collecting, transporting, and processing blood samples from a subject for cfDNA analysis, comprising:
collecting a blood sample from the subject at a first geographic location using a blood collection tube comprising an anticoagulant and an agent that inhibits the release of genomic DNA from white blood cells in the sample into the plasma component of the blood sample;
transporting the sample from the first geographic location collection to a second geographic location, wherein the sample is maintained at ambient temperature during transport;
preparing a digested cfDNA sample from the blood sample according to one of embodiments 1-19 at the second geographic location.

Embodiment 26. A method according to embodiment 25, wherein a time difference between collecting the blood sample and preparing the digested cfDNA sample is between about 8 hours and about 36 hours.

Embodiment 27. A method according to embodiment 26, wherein a time difference between collecting the blood sample and preparing the digested cfDNA sample is between about 8 hours and about 24 hours.

Embodiment 28. A method according to embodiment 26, wherein a time difference between collecting the blood sample and preparing the digested cfDNA sample is at least about 12 hours.

### EXAMPLES

### Example 1. Digestion Efficiency of Stabilized cfDNA Samples

Eleven individual clinical patient blood samples were collected in both standard lavender top EDTA blood collection tubes and Cell-Free BCT® blood collection tubes (Streck, Inc.). Each tube contained 10ml of blood. For each patient, tubes were collected and processed in replicates ≥2 per tube type. Prior to plasma separation, EDTA blood tubes were kept at 4° C. for 2 hr and Streck BCT blood tubes were maintained at ambient temperatures (4-40° C.) for 72 hr to represent shipping of blood samples to a central laboratory for processing.

Plasma separation was performed using double centrifugation according to established methods. cfDNA was extracted from samples using the QIAmp® circulating nucleic acid kit (Qiagen, Inc.). Extracted cfDNA was then subjected to either 2 hr (EDTA & Streck), or 16 hr (Streck only) digestion with the methylation-sensitive HinP1I endonuclease at 37° C., followed by qPCR amplification of 6 digestible genomic loci.

The level of marker-specific digestion was assessed by its amplification level relative to a non-digestible internal reference (IR) genomic locus, such that digestion of each marker was expressed as a deltaCq (dCq=[Cq of target locus]−[Cq of IR]). Overall MSRE digestion of a sample was then expressed as the sum of dCq across all 6 digestible genomic loci. A larger sum of dCq is indicative of better digestion.

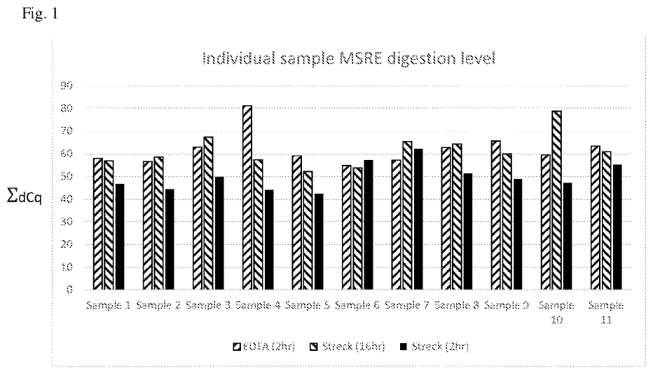

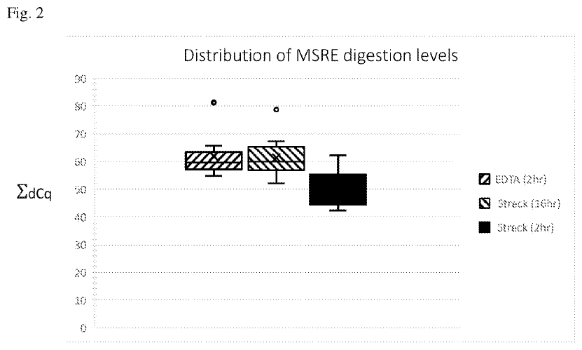

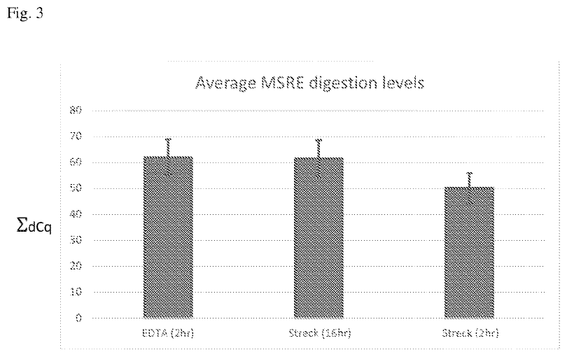

FIG. 1 shows MSRE digestion levels, expressed as the sum of dCq across all tube/digestion protocols, for all 11 samples is presented. FIG. 2 depicts these results grouped in a box-and-whisker plot, and FIG. 3 as a bar graph.

Comparing samples from a standard EDTA tube to samples maintained in a cfDNA-stabilizing blood collection tube, cfDNA stabilization resulted in a ˜23% lower digestion level (p<0.0005) in a 2 hr digestion. This loss of digestion efficiency was not resolved by either increasing the amount of endonuclease or by temperature adjustment. A similar result was seen in a double digest with another methylation-sensitive endonuclease.

Despite the fact that most routine restriction digests are incubated for one hour or less, and the HinPII endonuclease is reportedly active for only 4-8 hours (www.neb.com/tools-and-resources/usage-guidelines/restriction-endonucleases-survival-in-a-reaction), an extended (16 hr) digestion protocol was able to align the digestion of samples maintained in a cfDNA-stabilizing blood collection tube to that of cfDNA derived from conventional EDTA blood tubes (p<0.0005 comparing a 16 hr and a 2 hr digestion).

### General

The practice of the present invention will employ, unless otherwise indicated, conventional methods of chemistry, biochemistry, and molecular biology, within the skill of the art. Such techniques are explained fully in the literature. See, e.g., Methods In Enzymology (Academic Press, Inc.), Green & Sambrook (2012) Molecular Cloning: A Laboratory Manual, 4th edition (Cold Spring Harbor Press), Ausubel et al. (eds) Short protocols in molecular biology, 5th edition (Current Protocols), Molecular Biology Techniques: An Intensive Laboratory Course, (Ream & Field, eds., 1998, Academic Press), Wilson and Walker's Principles and Techniques of Biochemistry and Molecular Biology (Hodmann & Clokie, 2018), Basic Molecular Biology & Techniques—Recent Advances: Molecular Biology & Its Technique (Singh et al., 2021), etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%, and in certain embodiments x±5%, x±2%, or x±1%.

The word “substantially” does not exclude “completely” e.g. a composition which is “substantially free” from Y may be completely free from Y. Where necessary, the word “substantially” may be omitted from the definition of the invention.

The term “between” with reference to two values includes those two values e.g. the range “between” 10 mg and 20 mg encompasses inter alia 10, 15, and 20 mg.

Unless specifically stated, a method comprising a step of mixing two or more components does not require any specific order of mixing. Thus components can be mixed in any order. Where there are three components then two components can be combined with each other, and then the combination may be combined with the third component, etc.

The various steps of methods may be carried out at the same or different times, in the same or different geographical locations, e.g. countries, and by the same or different people or entities.

### REFERENCES

WO 2011/109529
WO 2013/123030
WO 2014/078913
WO 2015/169947
WO 2020/188561
WO 2022/107145 (PCT/IL2021/051382)
U.S. Pat. No. 10,801,060
US 2020/0283840
Khulan et al. (2006) Genome Res 16: 1046-55
Schmidt et al. (2017) Clinica Chimica Acta 469: 94-8
van Paemel et al. (2021) Epigenetics 16, 797-807
van Zogchel et al. (2021) JCO Precision Oncology 1738-1748
Wielscher et al. (2015) EBioMedicine 2:929-36
Zhao et al. (2010) Prenat Diagn 30:778-82

All documents and online information cited herein are incorporated by reference in their entirety.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. It will be readily apparent to a person skilled in the art that varying substitutions and modifications may be made to the invention disclosed herein without departing from the scope and spirit of the invention. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims:

1. A method of preparing a sample from a subject for methylation analysis, comprising:
processing a blood sample to obtain the plasma component of the blood sample, wherein the blood sample was collected using a blood collection tube comprising an anticoagulant and an agent that inhibits the release of genomic DNA from white blood cells in the sample into the plasma component of the blood sample;
isolating cell-free DNA (cfDNA) from the plasma component of the blood sample to provide a cfDNA sample; and
digesting the cfDNA sample with one or more methylation-sensitive restriction enzymes (MSREs) and/or one or more methylation-dependent restriction enzymes (MDREs) at a temperature between about 30° C. to about 45° C. for a digestion period of between about 8hours to about 18 hours to provide a digested cfDNA sample, wherein less than 25% of the DNA molecules present in the cfDNA sample are single stranded DNA molecules during the digesting step.

2. A method according to claim 1, wherein the digestion period is between about 8 hours to about 11 hours.

3. A method according to claim 1, wherein the digestion period is between about 9 hours to about 10 hours.

4. A method according to claim 1, further comprising inactivating the one or more MSREs and/or one or more MDREs following the digesting step to halt the digestion.

5. A method according to claim 4, wherein the inactivating comprises heating the digested cfDNA sample to about 65° C. for at least 20 minutes.

6. A method according to claim 1, wherein less than 5% of the DNA molecules present in the cfDNA sample are single stranded DNA molecules during the digesting step.

7. A method according to claim 1, wherein less than 1% of the DNA molecules present in the cfDNA sample are single stranded DNA molecules during the digesting step.

8. A method according to claim 1, wherein the cfDNA sample is treated with a single-strand specific DNase to reduce the number of DNA molecules present in the cfDNA sample that are single stranded DNA molecules.

9. A method according to claim 8, wherein the single-strand specific DNase is an Exonuclease I.

10. A method according to claim 1, wherein the use of the blood collection tube inhibits digestion of the cfDNA by the one or more MSREs and/or one or more MDREs as compared to the use of an ISO 6710: 1995 standard lavender closure EDTA blood collection tube.

11. The method of claim 10, wherein the inhibition of digestion of the cfDNA is not resolvable by increasing the concentration of the one or more MSREs and/or one or more MDREs.

12. A method according to claim 1, wherein the method further comprises amplifying at least one restriction locus in the digested cfDNA sample.

13. The method according to claim 12, wherein the digesting step and the amplifying step occur in the same vessel.

14. The method according to claim 13, wherein the one or more MSREs and/or one or more MDREs are divalent cation-dependent, and the free divalent cation concentration in the digested cfDNA sample is reduced before the amplifying step.

15. The method according to claim 14, wherein the free divalent cation concentration is reduced by dilution.

16. The method according to claim 14, wherein the free divalent cation concentration is reduced by adding a chelating agent.

17. A method according to claim 1, wherein the one or more MSREs and/or one or more MDREs comprise one or more of AciI, HinP1I, and HhaI.

18. A method according to claim 1, wherein the agent that inhibits the release of genomic DNA from white blood cells comprises formaldehyde, a formaldehyde-releasing reagent, or formalin.

19. A method according to claim 1, wherein the anticoagulant is potassium EDTA.

20. A method for analysing cfDNA from a subject, comprising:
preparing a digested cfDNA sample from the subject according to claim 1; and
performing real time PCR on the digested cfDNA.

21. A method for analysing cfDNA from a subject, comprising:
preparing a digested cfDNA sample from the subject according to claim 1; and
sequencing of the digested cfDNA.

22. A method for assessing methylation status of one or more CpG sites in cfDNA obtained from a subject, comprising:
preparing a digested cfDNA sample from the subject according to claim 1;
quantifying a degree of digestion at one or more of the one or more CpG sites.

23. A method for diagnosing the presence of absence of a cancer in a subject, comprising:
preparing a digested cfDNA sample from the subject according to claim 1;
assessing methylation status of one or more CpG sites in the digested cfDNA, wherein hypermethylation and/or hypomethylation of the one or more CpG sites is associated with the cancer.

24. A method for treating or managing a cancer in a subject, comprising:
diagnosing a presence of cancer in the subject by the method of claim 23; and
administering an anti-cancer treatment effective for the treatment of the cancer to the subject.

25. A method for collecting, transporting, and processing blood samples from a subject for cfDNA analysis, comprising:
collecting a blood sample from the subject at a first geographic location using a blood collection tube comprising an anticoagulant and an agent that inhibits the release of genomic DNA from white blood cells in the sample into the plasma component of the blood sample;
transporting the sample from the first geographic location collection to a second geographic location, wherein the sample is maintained at ambient temperature during transport;
preparing a digested cfDNA sample from the blood sample according to claim 1 at the second geographic location.

26-28. (canceled)
